(19) United States
(12) Patent Application Publication
(10) Pub. No.: US 2023/0172435 A1
(43) Pub. Date: Jun. 8, 2023

(54) ENDOSCOPE COMPANION DEVICES WITH LOCKING ELEMENTS

(71) Applicant: GI Scientific, LLC, Arlington, VA (US)

(72) Inventor: Scott Miller, Arlington, VA (US)

(21) Appl. No.: 17/995,291

(22) PCT Filed: Apr. 9, 2021
(86) PCT No.: PCT/US2021/026528

§ 371 Date: Sep. 30, 2022

Related U.S. Application Data
(60) Provisional application No. 63/007,461, filed on Apr. 9, 2020.

(51) Int. Cl. A61B 1/00; A61B 1/018; A61B 17/00

(52) U.S. Cl. CPC A61B 1/00137; A61B 1/018; A61B 1/00098; A61B 17/00234; A61B 2017/0034

(57) ABSTRACT

An endoscope companion or coupler device comprises a proximal end configured for attachment to a working end of an endoscope and a visualization section for allowing viewing of tissue. The coupler device includes an instrument channel, such as a working channel extension or an open passageway, having an open distal end and a proximal end that is configured for alignment with a working channel of the endoscope. The coupler device is configured to constrain and/or secure an instrument that has passed through the instrument channel to ensure that the instrument remains in place at the target site and/or to facilitate instrument exchange. The coupler device also provides a protective cover to reduce the ingress of debris, fluid, bacteria, or other unwanted matter from the working end of the endoscope which could lead to infection and decreased performance of the scope.

### CROSS-REFERENCE TO RELATED APPLICATIONS

This application claims the benefit of U.S. Provisional Application No. 63/007,461, filed Apr. 9, 2020, the entire disclosure of which is incorporated herein by reference for all purposes as if copied and pasted herein.

### FIELD

The present disclosure relates to companion or coupling devices for covering and at least partially sealing an optical element of an endoscope and mechanisms for constraining and/or locking instruments exiting the working channel of the endoscope.

### BACKGROUND

Recent advances in optical imaging technology have allowed many medical procedures to be performed today in a minimally invasive manner. The evolution of the more sophisticated, flexible scope with advanced visual capabilities has allowed access to regions deep within the human body that could only be achieved before with invasive surgical intervention. This modern day convenience has resulted in an increase in the demand for, as well as the number of, endoscopic, laparoscopic, arthroscopic, ophthalmoscopic, or other remote imaging visualization procedures performed every year in the U.S and globally. While these procedures are relatively safe, they are not without risks.

Endoscopy, for instance, is a procedure in which a lighted visualization device called an endoscope is inserted into the patient's body to look inside a body cavity, lumen, organ or in combination, for the purpose of examination, diagnosis or treatment. The endoscope may be inserted through a small incision or through a natural opening of the patient. In a bronchoscopy, the endoscope is inserted through the mouth, while in a sigmoidoscopy, the endoscope is inserted through the rectum. Unlike most other medical imaging devices, endoscopes are inserted directly into the organ, body cavity or lumen.

Today, most endoscopes are reused. This means that, after an endoscopy, the endoscope goes through a cleaning, disinfecting or sterilizing, and reprocessing procedure to be introduced back into the field for use in another endoscopy on another patient. In some cases, the endoscope is reused several times a day on several different patients.

While the cleaning, disinfecting and reprocessing procedure is a rigorous one, there is no guarantee that the endoscopes will be absolutely free and clear of any form of contamination. Modern day endoscopes have sophisticated and complex optical visualization components inside very small and flexible tubular bodies, features that enable these scopes to be as effective as they are in diagnosing or treating patients. However, the tradeoff for these amenities is that they are difficult to clean because of their small size, and numerous components. These scopes are introduced deep into areas of the body which expose the surfaces of these scopes to elements that could become trapped within the scope or adhere to the surface, such as body fluids, blood, and even tissue, increasing the risk of infection with each repeated use.

Endoscopes used in the gastrointestinal tract, such as forward viewing scopes, endoscopic ultrasound scopes (EUS) and duodenoscopes with side- viewing capability, have an added complexity in that they are in a bacteria rich environment. Typical gastroscopes, colonoscopes, duodenoscopes and EUS scopes have a camera lens, light and working channels with distal openings exposed to the patient environment. These elements of the scope all create cleaning issues, including the risk that bacteria finds its way into the working channel and other hard to clean locations on the scope. This provides an opportunity for bacteria to colonize and become drug resistant, creating the risk of significant illness and even death for a patient. This infection risk is also present in the cable mechanisms that are used to articulate instruments passing through the working channel and in other aspects of current scope designs. Moreover, in addition to the health risks posed by bacterial contamination and patient-to-patient cross-contamination, the accumulation of fluid, debris, bacteria, particulates, and other unwanted matter in these hard-to-clean areas of the scope also impact performance, shortening the useful life of these reusable scopes.

Another drawback with many existing endoscopes is that while the instruments passing therethrough may be articulated to reach a desired target area in the patient, they are not secured in place as they pass through the distal end of the endoscope. In particular, flexible instruments, such as guidewires and the like, have a tendency to shift position during the procedure and thus do not remain at the desired target area. In addition, instrument exchange can be challenging and time-consuming because it requires the operator to reposition the second instrument or guidewire, e.g., if the operator desires to exchange an instrument over a guidewire.

Accordingly, it is desirable to provide devices which serve as convenient accessories for currently existing endoscopes to reduce the risk of contamination and infection, while also improving the performance of the endoscope. It is particularly desirable to provide an accessory or companion device for endoscopes that allows the user to protect the working end from bacterial contamination, to enable instruments to exit out of the working end of the scope at different angles and to constrain or secure these instruments in place after they have been advanced to a particular target site in the patient.

### SUMMARY

The present disclosure provides a coupler device for use with an endoscope comprising a proximal end configured for attachment to a working end of the endoscope and a visualization section for allowing viewing of tissue or other matter. The coupler device includes an instrument channel that is in operational proximity to a working or biopsy channel in the endoscope when the proximal end is attached to the endoscope. The coupler device is configured to constrain and/or secure the instrument to ensure that the instrument remains in place at the target site and/or to facilitate instrument exchange. The coupler device also provides a protective cover to reduce the ingress of debris, fluid, bacteria, or other unwanted matter from the working end of the endoscope which could lead to infection and decreased performance of the scope.

The instrument may include a guidewire, an endoscopic mucosal resection instrument, needle injector, Foley catheter, bipolar or monopolar electrosurgical or ultrasonic devices, snares, endoscopic staplers and other clamping or sealing instruments, arterial lines, drainage catheters, peripherally inserted central catheters, and any other device configured to penetrate and/or navigate in the body. The instrument may be configured to advance through the working channel of the endoscope and the instrument channel of the coupler device.

In one aspect of the invention, the instrument channel of the coupler device is a working channel extension having an open distal end and a proximal end that is configured for attachment to a working or biopsy channel within the endoscope. The coupler device is configured to secure the instrument in place after it has passed through the working channel extension so that it does not move around at the target site in the patient. This novel feature of the present disclosure can be used by an operator to fixate an instrument and/or to fixate a guidewire while another instrument is exchanged over the guidewire.

In other embodiments, the instrument channel may be open passageway, cavity or channel within the optical coupler that allows the instrument to pass through the coupler device to the surgical site. In these embodiments, the coupler device includes mechanisms for securing the instrument within the passageway. In addition, the instrument(s) may be articulated by a variety of suitable means, such as cables, elevators, piezo electric materials, micro motors, organic semiconductors, electrically activated polymers or other sources of energy or power, that are either disposed within the coupler device, on or within the endoscope, or external to both and suitably coupled to the instrument(s).

In certain embodiments, the coupler device is configured to secure the instrument at a particular angle relative to the endoscope shaft while allowing longitudinal movement of the instrument at the fixed angle. This ensures that the instrument is advanced at a fixed angle relative to the optical coupler and the endoscope. Alternatively, the instrument may be removed and exchanged with another instrument that can be easily and quickly advanced to the same location in the patient, e.g., guidewire or instrument exchange.

In one embodiment, the optical coupler device or the endoscope may include an actuator coupled to a portion of the working channel extension. The actuator is configured to compress at least a portion of the working channel extension to reduce its internal diameter and constrain the instrument passing therethrough. In a preferred embodiment, the actuator translates or articulates the working channel extension against another fixed portion of the coupler device, thereby compressing at least a portion of the working channel extension. Alternatively, the actuator may, for example, comprise an elongate component, such as a cable, rod, or the like, attached to the working channel extension and configured to translate longitudinally to press or pull against one side of the working channel extension, thereby compressing its internal diameter.

In certain embodiments, the coupler device may include a stop member positioned to engage a portion of the working channel extension, such that the working channel extension is configured to at least partially compress upon engagement with the stop member, thereby reducing the internal diameter of the working channel extension to constrain an instrument passing therethrough. The stop member may be translated and/or articulated against the working channel extension or the working channel extension may be translated and/or articulated against the stop member. Alternatively, the working channel extension may be articulated relative to the stop member such that the working channel is at least partially compressed as it engages the stop member.

In certain embodiments, the working channel extension of the coupler device can be angularly adjustable by an elevator or cable passing through the endoscope. Alternatively, the coupler device may include its own actuator, such as an elevator, cable, or similar actuation means, for adjusting the working channel extension and thereby articulating instruments passing through the endoscope. The actuator may be powered by any suitable source of energy, such as a motor or the like. The source of energy may be coupled to the actuator either directly through the scope, or indirectly through magnetic, electric, or some other source of energy. The source of energy may be disposed within the coupler device, or it may be external to the coupler device (i.e., either disposed on the proximal end of the scope or external to the patient).

In one particular embodiment, the coupler device further comprises a locking element on an outer surface of the main body. The elevator (or other actuator) is configured to articulate the instrument and/or the working channel extension against the locking element to secure the instrument in place. The locking element may comprise an elongate body protruding from an outer surface of the main body and extending transverse to a longitudinal axis of the main body. The instrument and/or working channel is articulated in a generally longitudinal direction against the elongate body of the locking element. The elongate body may further comprise one or more protrusions extending therefrom to define a groove for securing the instrument. In certain embodiments, the locking element may include multiple locations or grooves for securing more than one instrument thereto.

In another aspect of the invention, the endoscope is a side- viewing scope such as a duodenum scope, endoscopic ultrasound scope (EUS) or the like. The side-viewing scope includes a working channel, a light source and a camera. The scope may further comprise an actuator for adjusting the angle of the working channel extension of the optical coupler. In one embodiment, the actuator comprises an elevator disposed within a distal end portion of the scope. In another embodiment, the actuator comprises a cable extending through the scope In these embodiments, the coupler device is configured to cooperate with the scope's actuator or cable to articulate instruments through the coupler device. In other embodiments, the coupler device includes its own actuator for articulating instruments, eliminating the need to have a scope with an elevator or cable actuator.

In one preferred embodiment, the coupler device comprises an elevator for articulating the working channel extension. The elevator is configured to move the working channel extension against a fixed portion of the coupler device, thereby compressing a portion of the working channel extension and securing the instrument therein.

The articulation aspect of the coupler device may include a locking feature or capability to affix the angle of exit in the working channel extension at a specific angle. For example, the specific angle of exit may be aimed at a specific point in the gastrointestinal tract, such as a biliary or pancreatic duct, or the angle of exit may be affixed so that a wire or other instrument inside the working channel temporarily cannot be advanced, locking the instrument in position temporarily to aid in the exchange of instruments or to improve navigation of the instrument temporarily.

In another aspect of the invention, the working channel extension or the coupler device elevator comprises a groove at, or near, its distal end for receiving an instrument therein. The groove is preferably shaped to facilitate securing the instrument to the working channel extension or the elevator. In certain embodiments, the groove comprise a substantially V-shape. In other embodiments, the groove may define other shapes, such as a U-shape, square, rectangular, triangular, conical or the like.

In certain embodiments, the working channel extension or the elevator further includes one or more protrusions, such as bevels, ridges, creases or other projections, extending into the elevator or the working channel extension. The protrusion(s) may, for example, comprise a pair of ridges that form the groove at the open distal end of the working channel extension or the elevator. The protrusions may also serve to narrow or stricture the working channel extension towards its distal end to guide the instrument into the groove. The groove may be configured to secure an instrument, such as a guidewire, in place relative to the optical coupler and/or the distal end portion of the endoscope. In certain embodiments, the groove has a surface material or shape designed to take advantage of a guidewire's reactive force to secure the guidewire within the locking element.

The protrusion(s) and/or groove may be formed integrally with the working channel extension or elevator, or they may be part of a separate component coupled to the inner surface of the working channel extension or elevator. In certain embodiments, the protrusion(s) and/or groove comprise a removable component that can be coupled to the working channel extension or elevator for certain portions of a procedure and then removed when no longer needed.

In another aspect of the invention, a kit for use in a procedure on a patient comprises an endoscope having a light, a camera lens and a working channel for receiving an instrument and a coupler device. The coupler device comprises a main body having a visualization section for allowing viewing of tissue by the endoscope and a proximal end configured for attachment to a distal end portion of the endoscope. The coupler further includes a passageway within the main body for allowing the instrument to pass from the endoscope working channel to a target site within the patient. The coupler device is configured to constrain and/or secure an instrument passing through the passageway.

In certain embodiments, the kit further comprises an endoscopic device configured for advancement through an opening into the patient. For purposes of this disclosure, an opening means natural orifice openings through any pre-existing, natural opening into the patient, such as the mouth, sinus, ear, urethra, vagina or anus, or any access port provided through a patient's skin into a body cavity, internal lumen (i.e., blood vessel), etc. or through incisions, and port-based openings in the patient's skin, cavity, skull, joint, or other medically indicated points of entry. The endoscopic device may also be configured to pass through a working or biopsy channel within the endoscope (i.e., through the same access port as the endoscope) and further through the working channel extension or other passageway, including an alternative articulating element, of the coupler device. Alternatively, the endoscopic device may be configured to pass through an opening that is separate from the endoscope access point.

In certain embodiments, the endoscopic instrument is a guidewire for use with the endoscope and the coupler device. The guidewire preferably comprises an elongate shaft sized for advancement through a working channel of the endoscope and the working channel extension or other articulating element of the coupler device.. The guidewire comprises a distal tip sized to advance into a relatively narrow body lumen of the patient, such as pancreaticobiliary tract or other body lumen.

The orientation of the guidewire may be adjusted by actuating the working channel extension or other articulating element of the coupler device relative to the endoscope shaft. Alternatively, the guidewire may be adjusted through a separate actuator on the proximal end of the guidewire, on or within the endoscope, the coupler device or external to both. In all of these embodiments, the coupler device is configured to secure the guidewire after it has been articulated to the desired position.

The coupler device may be provided as a single-use disposable accessory to an endoscope that provides the user with the ability to change the angle of exit of a device being advanced out of the working channel of an endoscope and to secure the device in place, without exposing the distal end of the scope to bacteria, debris, fluid and particulate matter. In some embodiments, the coupler device includes a working channel extension having an open distal end and a proximal end that is configured for attachment to the working channel of the endoscope. The working channel extension can provide a seal against the scope working channel, so instruments can be passed back and forth through the scope working channel and out the working channel extension of the coupler device without fluid and bacteria entering areas outside of the scope working channel. This seal is accomplished, in some embodiments, through an extension of the device working channel into the scope working channel, through a gasket on the end of the working channel extension, by way of a temporary glue, through pressure and the seal of the overall device against the distal end of the scope, through the selection of elastic and elastomeric materials, and other suitable and alternative means.

The working channel extension of the coupler device can be made of one or more materials with elastic properties. The materials can include biocompatible material(s) when the device is intended for medical applications, which may include, without limitation, elastic and elastomeric materials, as well as combinations of rigid and flexible materials, including silicone joined to polycarbonate and other materials joined to a biocompatible metal.

In some embodiments, the working channel extension of the coupler device may include an elastic biocompatible material that reduces the friction involving in passing devices through the working channel extension, which is joined to a biocompatible metal, such as a coil spring, hypotube, or braid, an additional elastic material that is joined to the biocompatible metal, to improve flexibility, reduce kinking and aid in sealing the working channel of the device against the endoscope' s working channel.

In some embodiments, the device allows the user to articulate the working channel of the device in the direction preferred by the user of the endoscope, so that a wire, catheter or other instrument being advanced down the working channel of the endoscope can direct the wire or catheter or other instrument in a preferred direction different than the angle at which the instrument would exit the endoscope if the coupler device was not in place or if an elevator in the scope is not used. This redirection of an instrument has the benefit of assisting with the navigation of the device, while not allowing fluid, debris, particulate matter, bacteria and other unwanted elements to enter hard to clean areas of the endoscope, especially at the distal end of the endoscope.

The benefits of the invention include allowing the physician to change the angle of exit, so that one or more devices can be turned to enter a particular body lumen, such as a biliary duct or pancreatic duct, or other hard to reach area, including in non-medical procedures and securing the instrument in place after it has reached the particular body lumen or area, while sealing the distal end of the scope to prevent infection and the intrusion of debris and particulate matter into interior elements of the scope that are hard to reach to effectively clean.

In some embodiments, the device may be formed of an optically clear material that covers the end of the endoscope and seals the end of the endoscope, allowing visualization of the endoscope' s camera without obscuring the view by the device. The optically clear material may also cover the endoscope' s light guide to allow the light projected by the endoscope to illuminate the field of view of the endoscope. In some embodiments, the optically clear material may include navigation markers to orient the user when visualizing tissue, such as markers to identify the relative position of the scope as the user visualizes the tissue through the optically clear material.

In embodiments, the optically clear material may also include other markers to guide the user with confirmation of the accurate placement of the optically clear material over the endoscope's camera and, if applicable, over the endoscope's light guide.

In some embodiments, the device may articulate instruments through the device through a cable in a sealed sheath that is attached to the flexible working channel extension in the coupler device, allowing the user to advance and retract the cable to move the working channel extension or other articulating element backward and forward to change the angle of exit from the flexible working channel in order to direct an instrument to a desired direction.

In some embodiments, the device may have multiple cables so the angle of exit can be articulated in multiple directions, including in different quadrants, unlike with the current endoscope elevators, which can only deflect and therefore redirect an instrument in a single axis due to the limited travel of endoscope elevators, which can only be raised or lowered, but not moved from side to side or articulated into other quadrants. In some embodiments, the cable(s) may be attached directly to the working channel extension or to other devices that can be articulated and cause the working channel extension to change its angle of exit, including, for example, a dowel underneath the working channel extension, but encased in the device that can be advanced forward and backward to move the working channel extension as the cable is advanced and retracted. In some embodiments, the articulation ability of the coupler device may be created with an elevator embedded in the coupler device, which is disposable and therefore thrown away after the procedure.

The articulation ability of the coupler device may also take place with elements that do not involve cables, including for example, piezo electric materials, micro motors, organic semiconductors, and electrically activated polymers. In some embodiments, the articulation ability of the coupler device may also take place with the transfer of force to the working channel extension or an embedded elevator through interlocking connectors that transfer force, wires that twist, slidable sheaths, and memory metals that change shape through the transfer of temperature. In some embodiments, the device includes a power connector or motors to deliver energy, including electromagnetic energy, to the device to cause a transfer in force to change the angle of exit from the coupler device as an instrument is passed through the device, or in advance of passing an instrument through the device. This transfer of force can include causing the device to rotate as it exits the working channel extension. The device may be navigated and articulated by the user directly, or as part of a robotic system in which the users input is translated through the system through various means, including cables, power connectors, motors, electromagnetic energy, slidable sheaths, haptics, computer-guided and directed input, and other means to direct and guide the device to its intended location, including to specific diagnosis and treatment objectives in a patient, or in non-medical applications, to a desired remote location.

In some embodiments, the device may be integrated into a scope and configured to be detachable and reusable for separate cleaning, including manual cleaning, in an autoclave, an ETO sterilizer, gamma sterilizer, and other sterilization methods.

The device may include a disposable or reusable control mechanism that attaches to the endoscope to articulate the distal end of the coupler device to change the angle of exit from the working channel extension of the coupler device. In some embodiments, this control mechanism may also lock the angle of exit of the working channel extension or the working channel extension may be locked through elements in the endoscope itself, such as the elements that articulate the endoscope' s elevator.

In some embodiments, the coupler device may cover the entire distal end of the endoscope, or may just cover hard to clean areas. In some embodiments, the coupler device may cover the distal end of the endoscope, or a portion thereof, or it may include a sheath attached to the coupler device which covers the entirety of the scope that is exposed to fluid, debris, particulate matter, bacteria and other unwanted elements.

It is to be understood that both the foregoing general description and the following detailed description are exemplary and explanatory only and are not restrictive of the disclosure. Additional features of the disclosure will be set forth in part in the description which follows or may be learned by practice of the disclosure.

### BRIEF DESCRIPTION OF THE DRAWINGS

The accompanying drawings, which are incorporated in and constitute a part of this specification, illustrate several embodiments of the disclosure and together with the description, serve to explain the principles of the disclosure.

FIG. 1 is a partial cross-sectional view of the proximal portion of a representative endoscope according to the present disclosure;

FIG. 2 is a perspective view of the distal end portion of a side- viewing endoscope according to the present disclosure;

FIGS. 3A and 3B are isometric views of an exemplary embodiment of the coupler device of the present disclosure in use with a duodenum scope.

FIGS. 4A and 4B show partial cutaway views of the coupler device and a duodenum scope of FIGS. 3A and 3B, respectively.

FIG. 5 shows another cutaway view of the coupler device and a duodenum scope of FIGS. 3A and 3B.

FIG. 6 shows still another cutaway view of the coupler device and a duodenum scope of FIGS. 3A and 3B.

FIG. 7 is a cutaway side view of the coupler device and a duodenum scope of FIGS. 3A and 3B in a first position.

FIG. 8 is a cutaway side view of the coupler device and a duodenum scope of FIGS. 3A and 3B in a second position.

FIG. 9 is a cutaway side view of the coupler device and a duodenum scope of FIGS. 3A and 3B in a third position.

FIG. 10 is an enlarged side view of the working channel extension with membrane of the coupler device of FIGS. 3A and 3B.

FIG. 11 is a top-down view of the coupler device of FIGS. 3A and 3B.

FIG. 12 is a cutaway view of another exemplary embodiment of a coupler device of the present disclosure.

FIG. 13 is a cutaway side view of the coupler device of FIG. 12.

FIG. 14 is a cutaway side view of the coupler device of FIG. 12 in use with a duodenum scope.

FIG. 15 is an enlarged side view of an exemplary embodiment of a working channel extension of the present disclosure.

FIG. 16 is another enlarged side view of the working channel extension of FIG. 15.

FIG. 17A is a perspective view of the working channel extension

of FIG. 15.

FIG. 17B shows the working channel extension of FIG. 17A in use with an instrument.

FIG. 18 is a perspective top-down view of the coupler device of

FIG. 3 with a locking feature.

FIG. 19 is a perspective view of another exemplary embodiment of a working channel extension of the present disclosure.

FIG. 20 is a cross-sectional side view of a working channel extension comprising a groove for constraining and/or securing an instrument.

FIG. 21 is a cutaway side view of an alternative embodiment of the coupler device for use with the working channel extension of FIG. 20.

FIG. 22A illustrates one portion of a coupler device having an elevator with a groove for constraining and/or securing an instrument therein.

FIG. 22B illustrates one portion of a coupler device with an elevator having a lateral stop member for constraining and/or securing an instrument.

FIG. 23 is a cutaway side view of an alternative embodiment of the coupler device for use with a cable for securing an instrument within the working channel extension.

FIGS. 24A and 24B illustrate an embodiment of a coupler device with a locking element on an outer surface of the main body.

FIGS. 25A and 25B illustrate another embodiment of a locking element for a coupler device according to the present disclosure.

FIGS. 26A and 26B illustrate yet another embodiment of a locking element for a coupler device according to the present disclosure.

### DETAILED DESCRIPTION

This description and the accompanying drawings illustrate exemplary embodiments and should not be taken as limiting, with the claims defining the scope of the present disclosure, including equivalents. Various mechanical, compositional, structural, and operational changes may be made without departing from the scope of this description and the claims, including equivalents. In some instances, well-known structures and techniques have not been shown or described in detail so as not to obscure the disclosure. Like numbers in two or more figures represent the same or similar elements. Furthermore, elements and their associated aspects that are described in detail with reference to one embodiment may, whenever practical, be included in other embodiments in which they are not specifically shown or described.

For example, if an element is described in detail with reference to one embodiment and is not described with reference to a second embodiment, the element may nevertheless be claimed as included in the second embodiment. Moreover, the depictions herein are for illustrative purposes only and do not necessarily reflect the actual shape, size, or dimensions of the system or illustrated components.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non- limiting, such that recitation of items in a list is not to the exclusion of other like items that can be substituted or added to the listed items.

While the following disclosure is primarily directed to a coupler device for an optical image endoscope, it should be understood that the features of the presently described kit may be readily adapted for use with a variety of reusable or disposable endoscopic scopes, instruments and devices.

The term “endoscope” in the present disclosure refers generally to any scope used on or in a medical application, which includes a body (human or otherwise) and includes, for example, a laparoscope, duodenoscope, endoscopic ultrasound scope, arthroscope, colonoscope, bronchoscopes, enteroscope, cystoscope, laparoscope, laryngoscope, sigmoidoscope, thoracoscope, cardioscope, and saphenous vein harvester with a scope, whether robotic or non-robotic.

When engaged in remote visualization inside the patient's body, a variety of scopes are used. The scope used depends on the degree to which the physician needs to navigate into the body, the type of surgical instruments used in the procedure and the level of invasiveness that is appropriate for the type of procedure.

For example, visualization inside the gastrointestinal tract may involve the use of endoscopy in the form of flexible gastroscopes and colonoscopes, endoscopic ultrasound scopes (EUS) and specialty duodenum scopes with lengths that can run many feet and diameters that can exceed 1 centimeter. These scopes can be turned and articulated or steered by the physician as the scope is navigated through the patient.

Many of these scopes include one or more working channels for passing and supporting instruments, fluid channels and washing channels for irrigating the tissue and washing the scope, insufflation channels for insufflating to improve navigation and visualization and one or more light guides for illuminating the field of view of the scope.

Smaller and less flexible or rigid scopes, or scopes with a combination of flexibility and rigidity, are also used in medical applications. For example, a smaller, narrower and much shorter scope is used when inspecting a joint and performing arthroscopic surgery, such as surgery on the shoulder or knee. When a surgeon is repairing a meniscal tear in the knee using arthroscopic surgery, a shorter, more rigid scope is usually inserted through a small incision on one side of the knee to visualize the injury, while instruments are passed through incisions on the opposite side of the knee. The instruments can irrigate the scope inside the knee to maintain visualization and to manipulate the tissue to complete the repair

Other scopes may be used for diagnosis and treatment using less invasive endoscopic procedures, including, by way of example, but not limitation, the use of scopes to inspect and treat conditions in the lung (bronchoscopes), mouth (enteroscope), urethra (cystoscope), abdomen and peritoneal cavity (laparoscope), nose and sinus (laryngoscope), anus (sigmoidoscope), chest and thoracic cavity (thoracoscope), and the heart (cardioscope). In addition, robotic medical devices rely on scopes for remote visualization of the areas the robotic device is assessing and treating.

These and other scopes may be inserted through natural orifices (such as the mouth, sinus, ear, urethra, anus and vagina) and through incisions and port- based openings in the patient's skin, cavity, skull, joint, or other medically indicated points of entry. Examples of the diagnostic use of endoscopy with visualization using these medical scopes includes investigating the symptoms of disease, such as maladies of the digestive system (for example, nausea, vomiting, abdominal pain, gastrointestinal bleeding), or confirming a diagnosis, (for example by performing a biopsy for anemia, bleeding, inflammation, and cancer) or surgical treatment of the disease (such as removal of a ruptured appendix or cautery of an endogastric bleed).

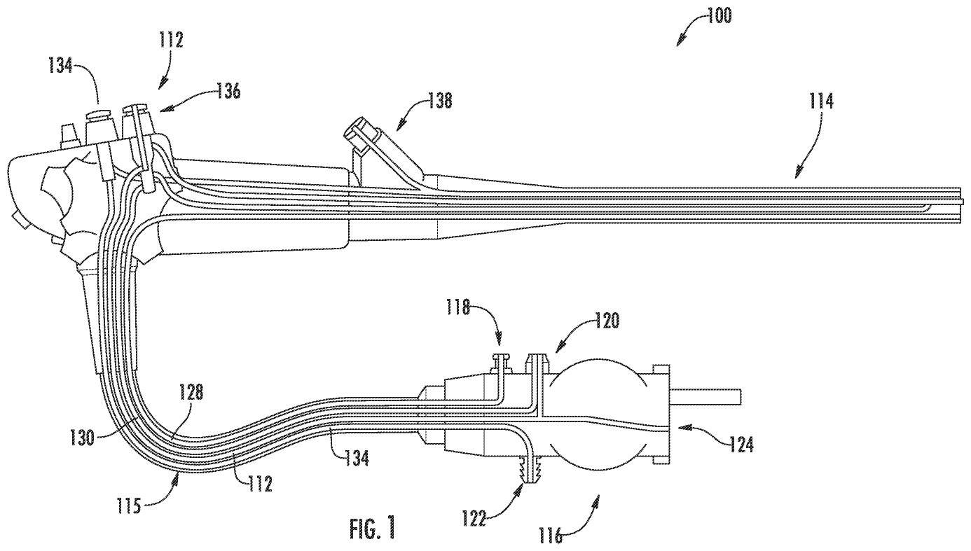

Referring now to FIG. 1, the devices, systems and methods of the present disclosure may include an optical viewing endoscope of the type described above. A representative endoscope 100 for use with the present disclosure includes a proximal handle 112 adapted for manipulation by the surgeon or clinician coupled to an elongate shaft 114 adapted for insertion through a natural orifice or an endoscopic or percutaneous penetration into a body cavity of a patient. Endoscope 100 further includes a fluid delivery system 116 coupled to handle 112 via a universal cord 115. Fluid delivery system 116 may include a number of different tubes coupled to internal lumens within shaft 114 for delivery of fluid(s), such as water and air, suction, and other features that may be desired by the clinician to displace fluid, blood, debris and particulate matter from the field of view. This provides a better view of the underlying tissue or matter for assessment and therapy. In the representative embodiment, fluid delivery system 116 includes a water-jet connector 118, water bottle connector 120, a suction connector 122 and an air pipe 124. Water-jet connector 118 is coupled to an internal water-jet lumen 126 that extends through handle 112 and elongate shaft 114 to the distal end of endoscope 100. Similarly, water jet connector 118, water bottle connector 120, suction connector 122 and air pipe 124 are each connected to internal lumens 128, 130, 132, 134 respectively, that pass through shaft 114 to the distal end of endoscope 100.

Endoscope 100 may further include a working channel (not shown) for passing instruments therethrough. The working channel permits passage of instruments down the shaft 114 of endoscope 100 for assessment and treatment of tissue and other matter. Such instruments may include cannula, catheters, stents and stent delivery systems, papillotomes, wires, other imaging devices including mini- scopes, baskets, snares and other devices for use with a scope in a lumen.

Proximal handle 112 may include a variety of controls for the surgeon or clinician to operate fluid delivery system 116. In the representative embodiment, handle 112 include a suction valve 135, and air/water valve 136 and a biopsy valve 138 for extracting tissue samples from the patient. Handle 112 will also include an eyepiece (not shown) coupled to an image capture device (not shown), such as a lens and a light transmitting system. The term “image capture device” as used herein also need not refer to devices that only have lenses or other light directing structure. Instead, for example, the image capture device could be any device that can capture and relay an image, including (i) relay lenses between the objective lens at the distal end of the scope and an eyepiece, (ii) fiber optics, (iii) charge coupled devices (CCD), (iv) complementary metal oxide semiconductor (CMOS) sensors. An image capture device may also be merely a chip for sensing light and generating electrical signals for communication corresponding to the sensed light or other technology for transmitting an image. The image capture device may have a viewing end — where the light is captured. Generally, the image capture device can be any device that can view objects, capture images and/or capture video.

In some embodiments, endoscope 100 includes some form of positioning assembly (e.g., hand controls) attached to a proximal end of the shaft to allow the operator to steer the scope. In other embodiments, the scope is part of a robotic element that provides for steerability and positioning of the scope relative to the desired point to investigate and focus the scope.

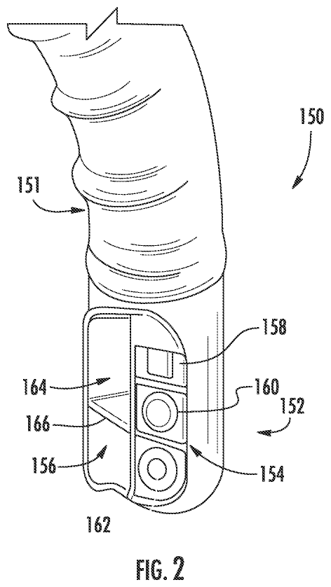

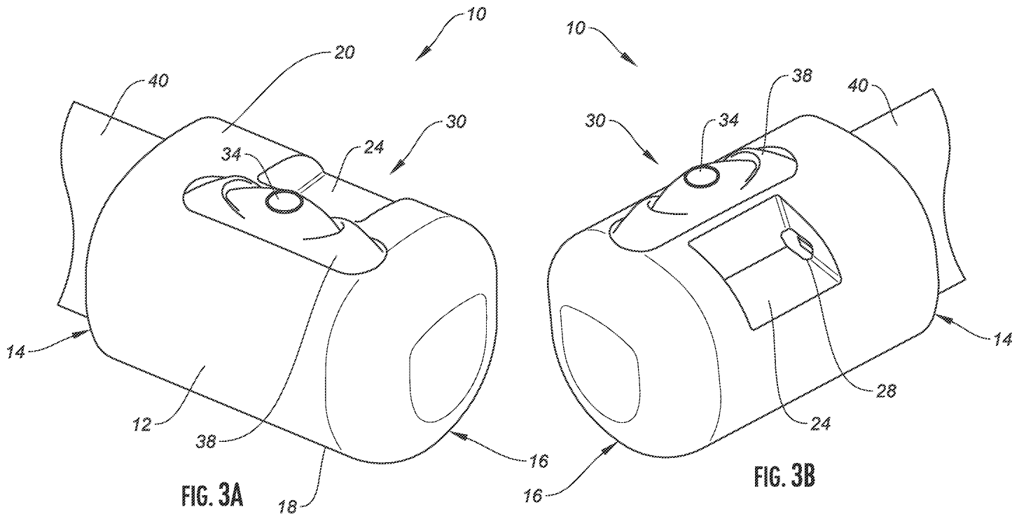

Referring now to FIG. 2, a distal end portion of a side viewing endoscope 150 (e.g., a duodenoscope or EUS) will now be described. As shown, scope 150 includes an elongate flexible shaft 151 with distal end portion 152 having a viewing region 154 and an instrument region 156, both of which face laterally or to the side of the longitudinal axis of shaft 151. Viewing region 154 includes an air nozzle port 158, a camera lens 160 and a light source 162 for providing a view of the surgical site in the patient. Instrument region 156 includes an opening 164 coupled to a working channel (not shown) within shaft 151 of scope 150. Opening 164 is configured to allow passage of instruments from the working channel of scope 150 to the surgical site. Scope 150 also preferably includes an articulation mechanism for adjusting the angle that the instruments pass through opening 164. In the exemplary embodiment, the articulation mechanism comprises an elevator 166, although it will be recognized by those skilled in the art that the articulation mechanism may include a variety of other components designed to articulate the instrument angle, such as a cable extending through shaft 151 or the like.

FIGS. 3A and 3B illustrate an exemplary embodiment of a coupler device 10 of the present disclosure. The coupler device 10 serves as an accessory component for currently existing endoscopes. The device seals and covers infection prone areas of the scope to prevent ingress of debris, fluid, or other unwanted matter that could lead to bacterial contamination and decreased performance of the scope.

In certain embodiments, the coupler device 10 provides a flexible working channel for instruments to be inserted into the scope. The flexible working channel can be angularly adjustable with ease. As shown, in the preferred embodiments, the coupler device 10 may be used with a duodenum scope 40 or other side-viewing scope instrument. It is understood, of course, that the coupler device 10 may be adapted for use with end viewing scopes as well. In addition, the coupler device 10 of the present disclosure can be used with all types of scopes for different medical applications. The duodenum scope 40 shown here is merely for illustrative purposes.

Of course, it will be recognized that the instruments passing through the scope may be articulated by a variety of different mechanism. For example, in some embodiments, the device may have multiple cables so the angle of exit can be articulated in multiple directions, including in different quadrants, unlike with the current endoscope elevators, which can only deflect and therefore redirect an instrument in a single axis due to the limited travel of endoscope elevators, which can only be raised or lowered, but not moved from side to side or articulated into other quadrants. In some embodiments, the cable(s) may be attached directly to the working channel extension or to other devices that can be articulated and cause the working channel extension to change its angle of exit, including, for example, a dowel underneath the working channel extension, but encased in the device that can be advanced forward and backward to move the working channel extension as the cable is advanced and retracted. In some embodiments, the articulation ability of the coupler device may be created with an elevator embedded in the coupler device, which is disposable and therefore thrown away after the procedure.

The articulation ability of the coupler device may also take place with elements that do not involve cables, including for example, piezo electric materials, micro motors, organic semiconductors, and electrically activated polymers. In some embodiments, the articulation ability of the coupler device may also take place with the transfer of force to the working channel extension or an embedded elevator through interlocking connectors that transfer force, wires that twist, slidable sheaths, and memory metals that change shape through the transfer of temperature. In some embodiments, the device includes a power connector or motors to deliver energy, including electromagnetic energy, to the device to cause a transfer in force to change the angle of exit from the coupler device as an instrument is passed through the device, or in advance of passing an instrument through the device. This transfer of force can include causing the device to rotate as it exits the working channel extension. The device may be navigated and articulated by the user directly, or as part of a robotic system in which the users input is translated through the system through various means, including cables, power connectors, motors, electromagnetic energy, slidable sheaths, haptics, computer-guided and directed input, and other means to direct and guide the device to its intended location, including to specific diagnosis and treatment objectives in a patient, or in non-medical applications, to a desired remote location.

As FIGS. 3A and 3B illustrate, the coupler device 10 may comprise a main body 12, proximal end 14 and distal end 16, lower surface 18 and upper surface 20. The proximal end 14 attaches onto a working end of a duodenum scope 40, extending the working end portion of the scope 40. The upper surface 20 may include a lens and light guide 24 and a scope washer opening 28, which is used to push fluid across the scope camera to wash debris off the camera and is also used to push air across the camera to dry the camera and insufflate the patient's gastrointestinal tract. Upper surface 20 may further include an open area over lens and light guide 24 and scope washer opening 28 to facilitate viewing the surgical site and to allow egress of fluid from scope washer opening 28 into the surgical site (and/or egress of air that may be passed over light guide 24 to dry the camera or that may be passed into the surgical site to insufflate a portion of the site). In addition, the upper surface 20 includes a flexible working channel region 30 that includes a flexible working channel extension 34 that is surrounded by a flexible membrane 38. This flexible membrane 138 serves as a protective hood or covering for the working end of the coupler device 10, providing for flexible articulation while sealing out debris, fluid, bacteria or other unwanted matter.

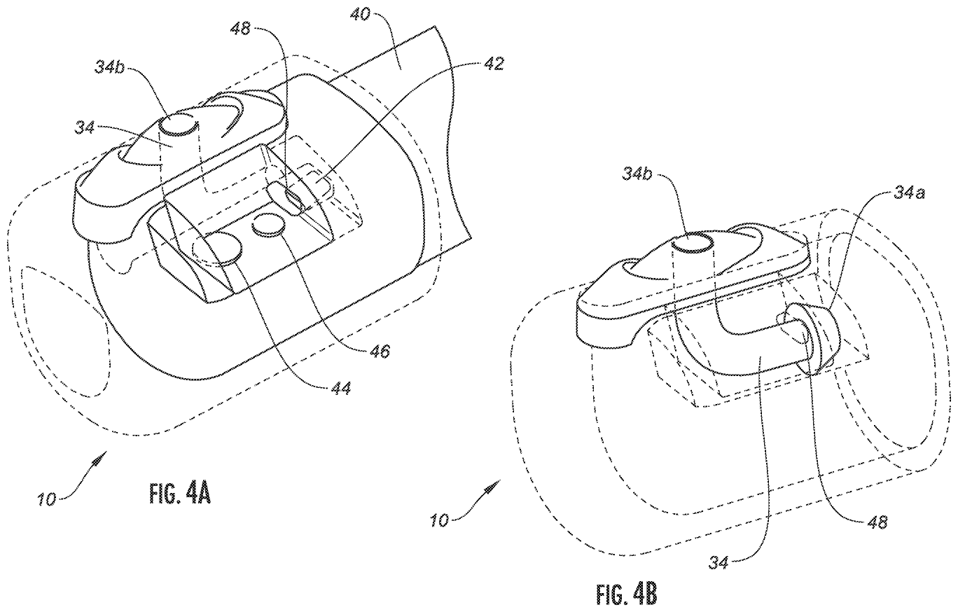

As shown in FIGS. 4A and 4B, the duodenum scope 40 may comprise a light guide 44, lens 46 and washer opening 48. The coupler device 10 cooperates with each of these components of the scope 40 to provide a fully functioning scope. The coupler device 10 does not interfere with the scope's ability to emit a clear image, but instead reduces the risk of contamination with each use. This benefit is achieved by providing a coupler device 10 which attaches to the working end components of the scope 40, and seals around the working end.

Coupler device 10 may comprise one or more optical layers configured to reduce an amount of reflected light from the surface and/or to inhibit condensation of water droplets on the visualization section, or both. Reducing the glare and/or fogging of the visualization section significantly improves the surgeon's view of the target site through coupler device 10. A more complete description of suitable optical layers for use with the present invention can be found in U.S. Provisional

Application Serial No. 62/949,238, filed December 17, 2019, the complete disclosure of which is hereby incorporated herein by reference in its entirety as if copied and pasted herein.

Coupler device 10 may further include one or more sensors on, or within, an outer surface of the main body of the coupler device. The sensors are configured to detect a physiological parameter of tissue around the outer surface of the main body of the coupler device. The physiological parameter may include, for example, a temperature of the tissue. a dimension of the tissue, a depth of the tissue, tissue topography, tissue biomarkers, tissue bioimpedance, temperature, PH, histological parameters or another parameter that may be used for diagnosing a medical condition. Coupler device 10 may be part of an overall system for analyzing, diagnosing, monitoring, treating and/or predicting tissue conditions by detecting and objectively quantifying images and physiological parameters in a patient's body, such as the size, depth and overall topography of tissue, tissue biomarkers, tissue bioimpedance, temperature, PH, histological parameters, lesions or ulcers, bleeding, stenosis, pathogens, abnormal or diseased tissue, cancerous or precancerous tissue and the like. A more complete description of such a system can be found in commonly- assigned, U.S. Provisional Application No. 63/003,656, filed April 1, 2020, the complete disclosure of which is hereby incorporated herein by reference in its entirety as if copied and pasted herein.

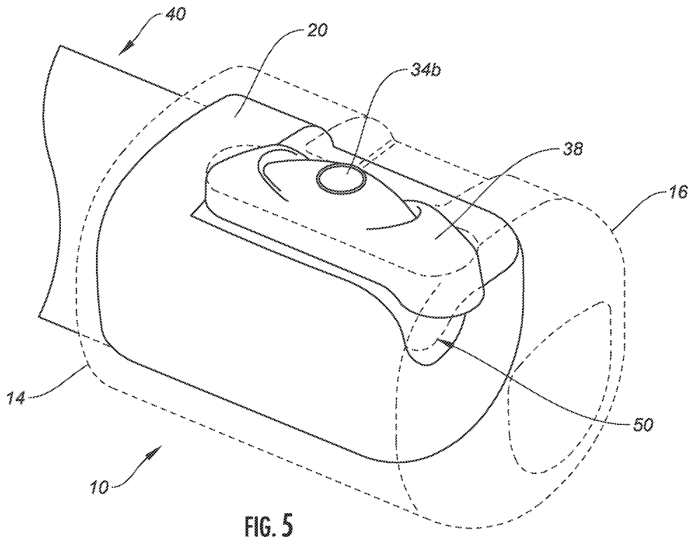

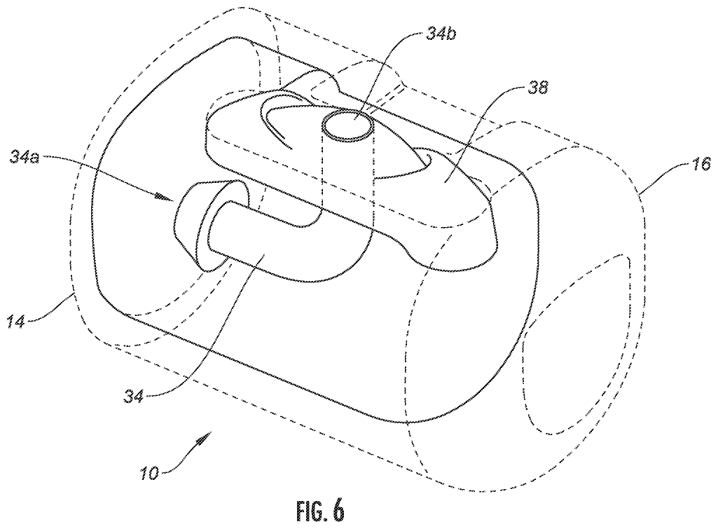

As further shown in FIGS. 3A, 3B, 4A, 4B, 5 and 6, the coupler device 10 provides an extension of the scope's working channel 42. The working channel extension 34 of the coupler device 10 in FIG. 3 is flexible and may contact the scope's working channel 42 by a sealed connection, as shown in FIG. 6, at the proximal end 34a of the working channel extension. The distal end 34b of the working channel extension 34 serves as an exit portal for instruments to pass through the scope 40 to reach different areas of the body.

Additionally, the coupler device 10 provides a further seal around the elevator 50 of the scope. Because the coupler device 10 seals the elevator 40, risk of debris influx, fluids, bacteria and other matter build up behind the elevator and working channel is reduced significantly. This influx of debris, bacteria and other matter is believed to be the reason for drug resistant infections with current scopes today. While preventing influx, the coupler device 10 advantageously maintains flexibility to move the working channel extension 34.

In use, the scope's working channel extension 34 permits passage of instruments down the scope working channel 42 and through and out the working channel extension 34 of the device 40 for assessment and treatment of tissue and other matter. Such instruments may include cannula, catheters, stents and stent delivery systems, papillotomes, wires, other imaging devices including mini-scopes, baskets, snares and other devices for use with a scope in a lumen. This working channel extension 34 is flexible enough that the elevator 50 of the scope 40 can raise and lower the working channel extension 34 so that instruments can be advanced down and out of the working channel extension distal end (or exit portal) 34b of the scope 40 at various angles, or be raised and lowered by a cable or other means to articulate the working channel extension 34.

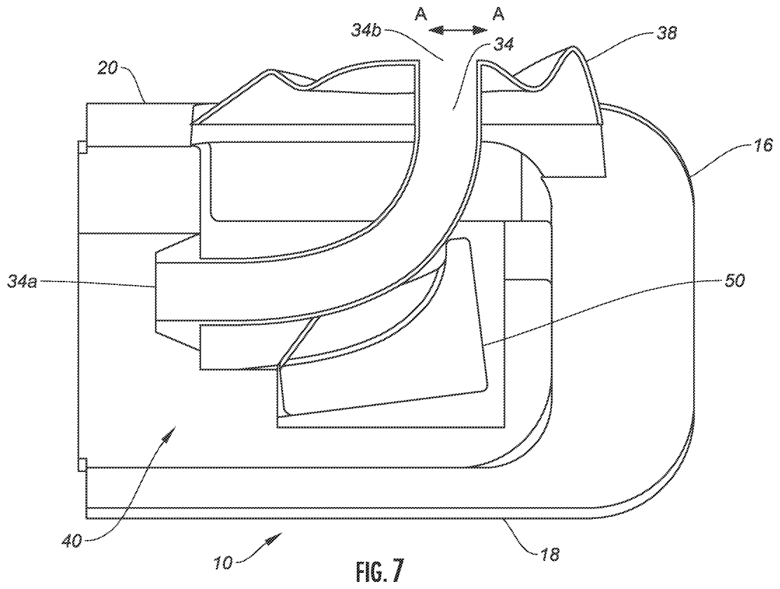

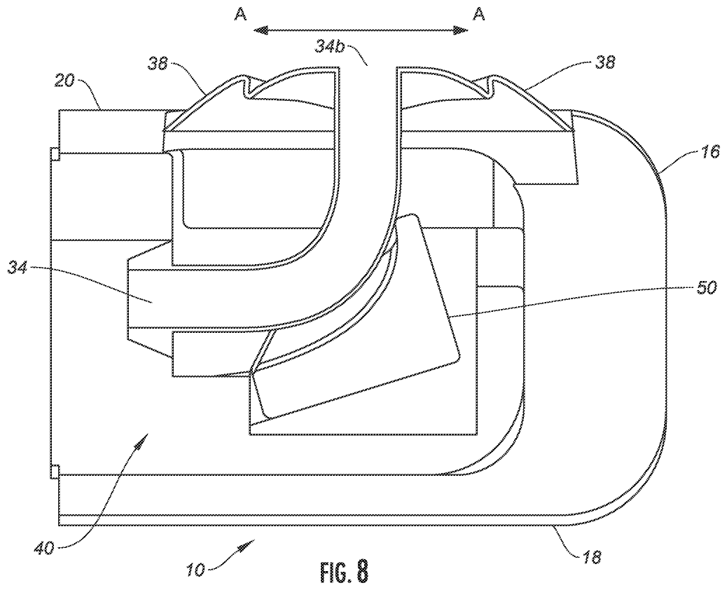

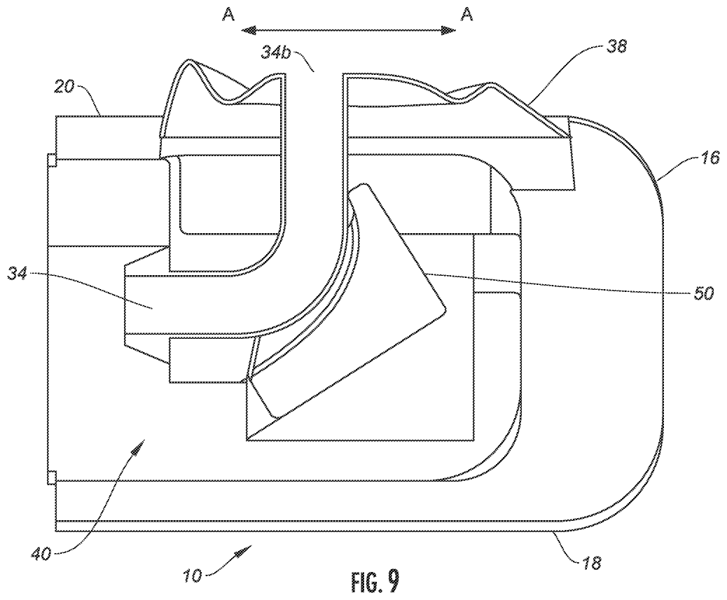

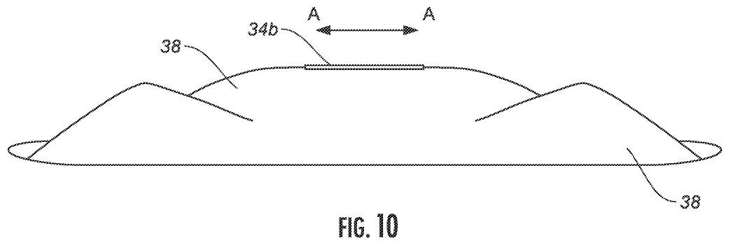

As FIGS. 7 to 9 illustrate, in use when the elevator 50 of the scope 40 is actuated, the flexible working channel extension 34 of the coupler device moves or adjusts to this actuation, along the direction A—A. In FIG. 7, the elevator 50 is raised slightly, creating a hinged ramp or shoulder that pushes the working channel extension 34 a corresponding angle and shifts the exit portal or distal end 34b of the working channel extension to the left. In FIG. 8 the elevator is raised higher than in FIG. 7, such that the distal end 34b of working channel extension 34 is likewise shifted further to the left in comparison to FIG. 7, while FIG. 9 shows the elevator 50 raised even higher and the distal end 34b of working channel extension 34 moved to the left even further in comparison to FIGS. 7 and 8.

Referring now to FIG. 10, the ability of the distal end 34b of working channel extension 34 to shift along the width of the working channel region 30 of the coupler device 10 is in part due to the fact that the distal end 34b is itself attached to a flexible membrane 38. This flexible membrane 38 comprises a plurality of loose folds or creases, allowing the excess material to stretch and bend as the elevator actuation forces the working channel extension to bend and shift in response. In addition, the flexible membrane 38 acts as a protective cover or hood for the working channel region 38, preventing the ingress of fluids, debris, or other unwanted matter from getting inside the scope 40 and causing a bacterial contamination or the infusion of other unwanted fluid, debris or particulate matter.

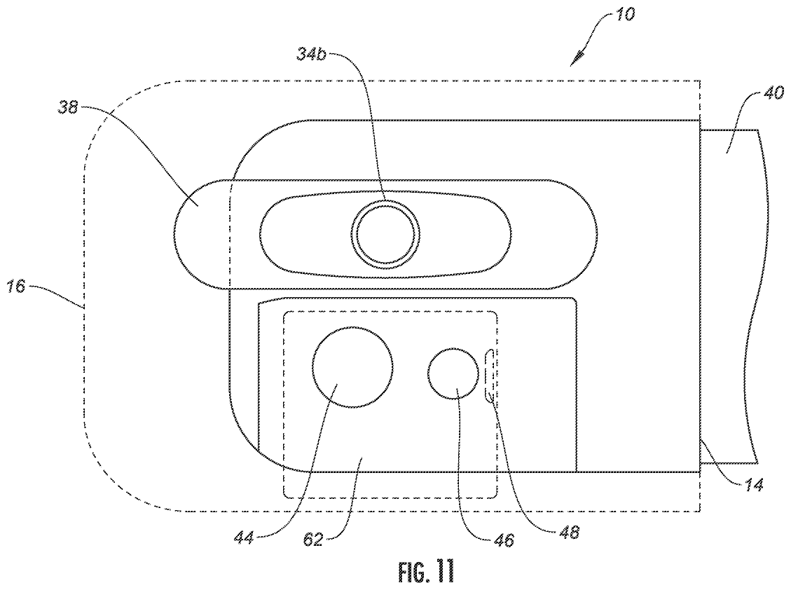

It is contemplated that the coupler device 10 of the present disclosure may be configured for single, disposable use, or it may be configured for reuse. The coupler device 10 may be made of any biocompatible material, such as for example, silicone or another elastic or polymeric material. In addition, the material may be transparent. As shown in FIG. 11, the coupler device 10 may be formed of a transparent material to provide a transparent covering of the scope camera and light source, thereby allowing unhindered performance of the scope 40.

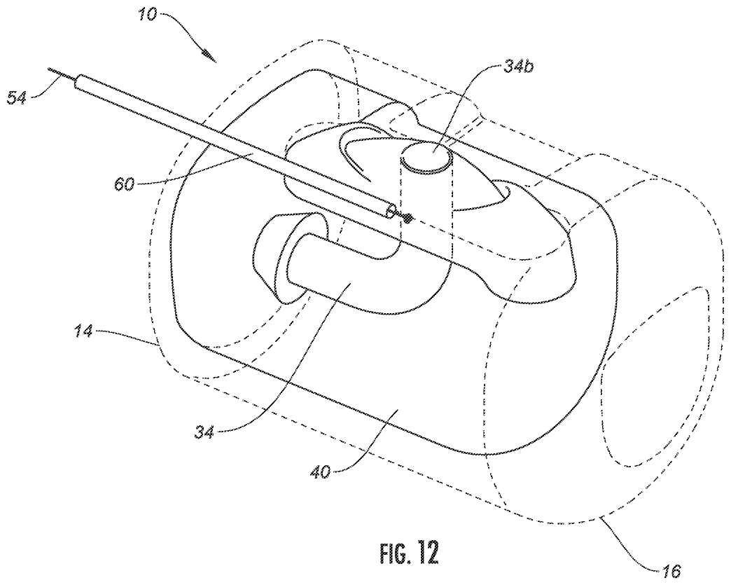

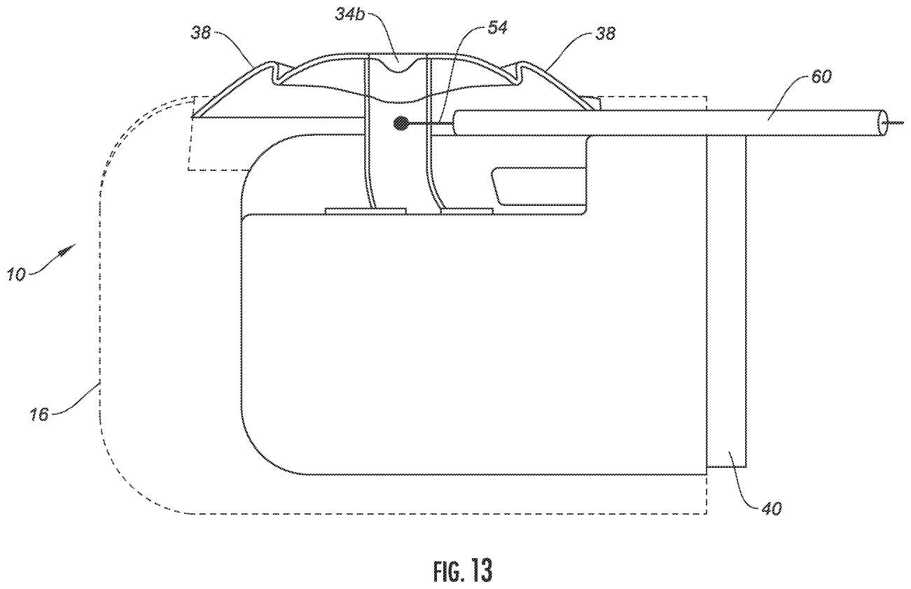

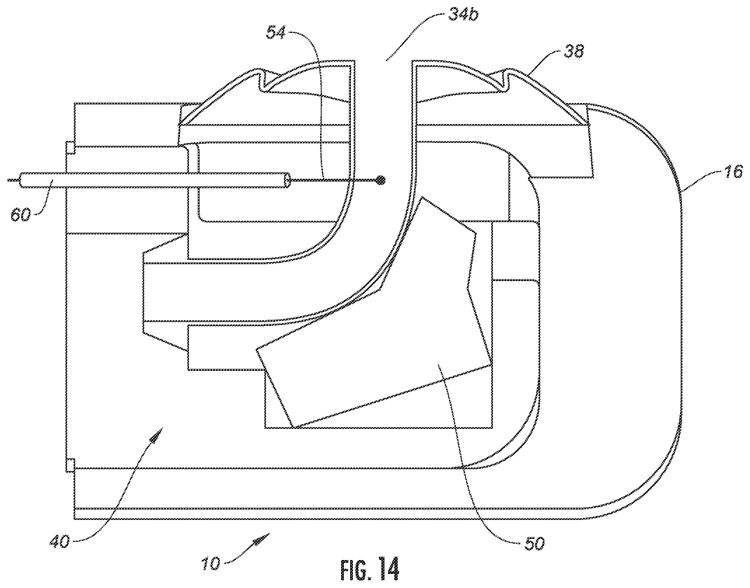

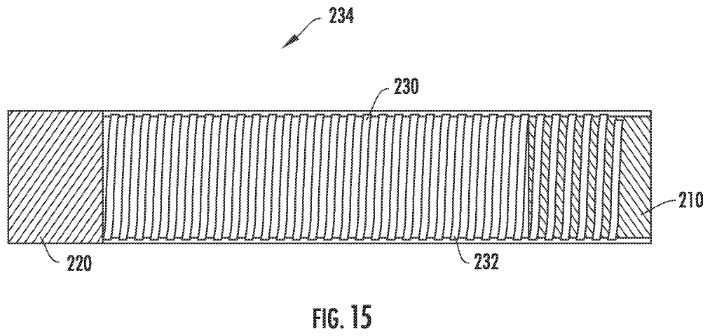

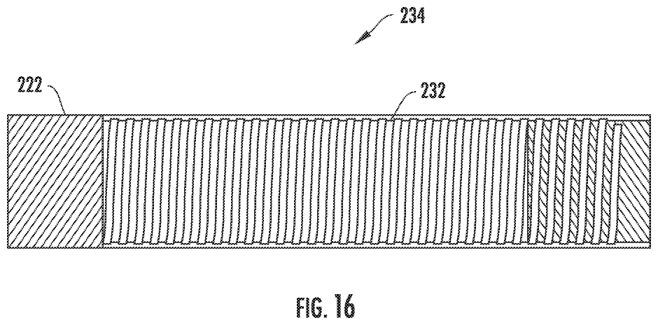

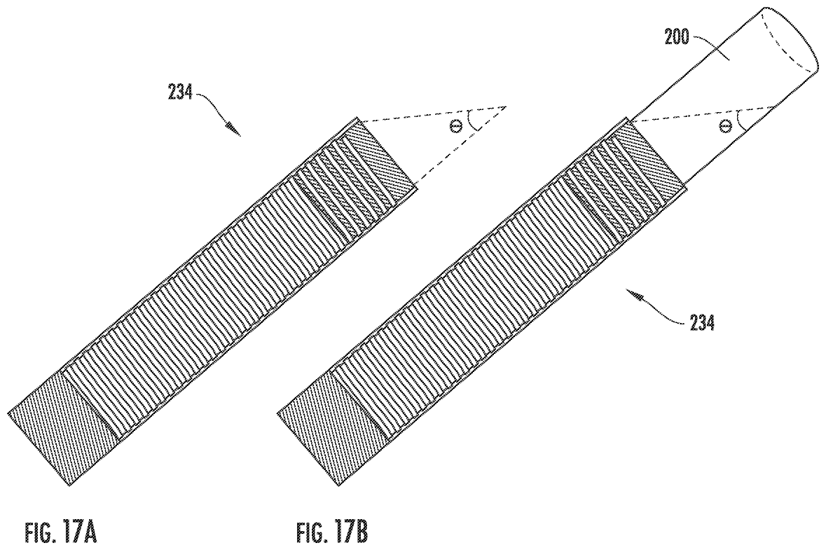

FIGS. 12 to 14 show another exemplary embodiment of a coupler device 10 of the present disclosure. In this embodiment, the coupler device 10 is adapted for use with scopes that are actuated by cable and eliminates the need for the elevator component. As illustrated, the coupler device 10 maintains the same structural features as previously described, but now includes a further disposable external sheath 60 that can receive an interior actuating cable 54 of the scope. This cable 54 can be detached from the elevator and reattached to the flexible working channel extension 34 of the coupler device 10. The elevator is no longer needed in this embodiment, as actuation of the cable effects movement of the working channel extension 34. The external sheath 60 may be configured to attach directly to the scope 40, such as by winding around the outside of the scope or by a friction fit connection. In embodiments, multiple cables may be included in one or more sheaths to provide for articulation in other quadrants than the single axis articulation with elevators in current duodenoscopes.

In other embodiments, the coupler device 10 may also include a closable port (i.e., self-sealing) that allows for the injection of anti-adhesion, anti- bacterial, anti-inflammatory or other drug or infusible matter that prevents the adherence or colonization of bacteria on the scope. An applicator may be provided that is integrated into the coupler device 10 with a port for delivery of the infusible matter. Alternatively, the applicator may be separate from the coupler device 10 and applied to the distal end of the scope 40. The infusible matter may include forms of silver, including in a gel or other solution, platinum, copper, other anti-adhesion, anti- bacterial, anti-inflammatory or other drug or infusible matter that is compatible with the scope and coupler device materials and biocompatible for patient use.

In one exemplary embodiment, the device includes an anti- infective material. In another exemplary embodiment, the device includes an anti- infective coating. In still another embodiment, the device includes a coating that is hydrophobic. In yet another embodiment, the device is superhydrophobic. In even still another embodiment, the device is anti-infective and hydrophobic. Further yet in another embodiment, the device is anti-infective and superhydrophobic. In further still another exemplary embodiment, anti-inflammatory coatings are incorporated into the device. In other embodiments, the anti-inflammatory coating may be hydrophilic.

In one exemplary embodiment, the device 10 may include a silver ion coating. In another embodiment, the device 10 may have a silver hydrogel applied, infused, or made part of the device 10 in the area that covers or goes around the scope elevators. In addition to silver having antimicrobial properties, silver can also conduct electricity. Thus, in still another embodiment, the device 10 may include an electrical wire or other power transmission point to enable the creation of an electric field across the silver ion coating to improve the ability of the silver ion coating to prevent infection. In some embodiments, the electrical wire or other power transmission point may also apply to other antimicrobial and conductive materials, including platinum and copper.

FIGS. 15 and 16 show another embodiment of the working channel extension 234 of the present disclosure. As contemplated, the working channel extensions may comprise a combination of different materials. For example, as shown in FIG. 15, the working channel extension 234 may be formed of multiple elastic materials joined to a biocompatible metal. In some embodiments, one of the elastic materials may be PTFE and another elastic material may be a biocompatible elastic material that covers the biocompatible metal. In the example of FIG. 15, the working channel extension 234 may comprise an inner elastic material 210 and an outer elastic material. The outside of the working channel extension 234 may include a biocompatible metal 230, which may take the form of a coil or winding 232. In one embodiment, the biocompatible metal may be encapsulated by one or more of the elastic materials.

In FIG. 16, the outer biocompatible elastic material 220 is formed to create a gasket 222 to seal the proximal end of the working channel extension 234 against the working channel of an endoscope, creating a seal to prevent the intrusion of unwanted bacteria, biomatter and other material into this sealed area.

In FIG. 17A, a working channel extension 234 is shown with an adjustable angle of exit 0 for locking an instrument 200 in place. In this embodiment, when the angle of exit 0 is adjusted, it creates compressive force in the working channel 234, locking an instrument 200 in place, as shown in FIG. 17B. This can be used to fixate an instrument while a wire is advanced through the instrument, or to fixate a wire, while a second instrument is exchanged over the wire.

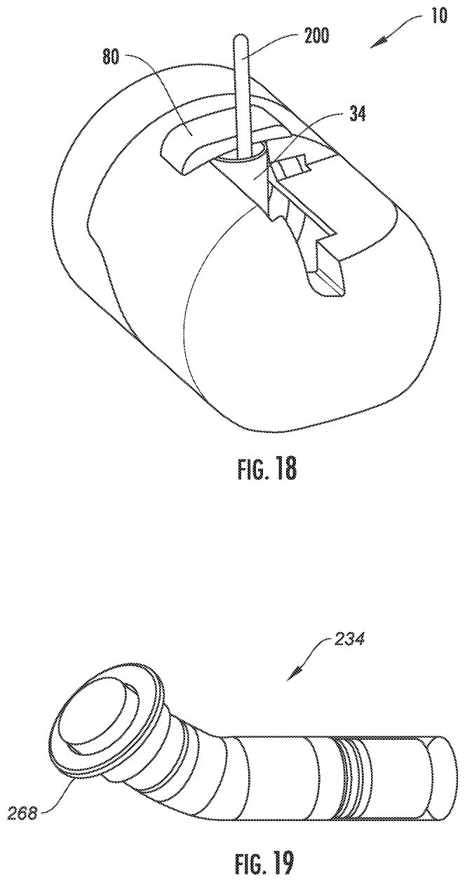

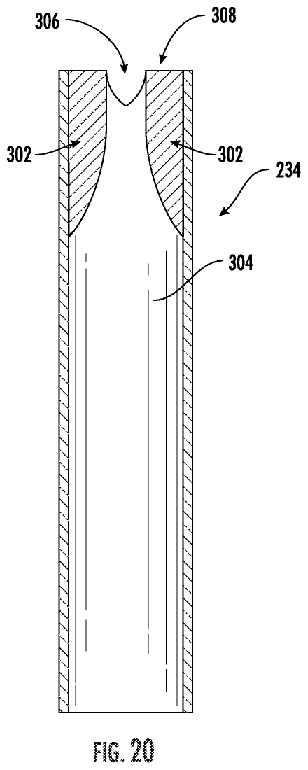

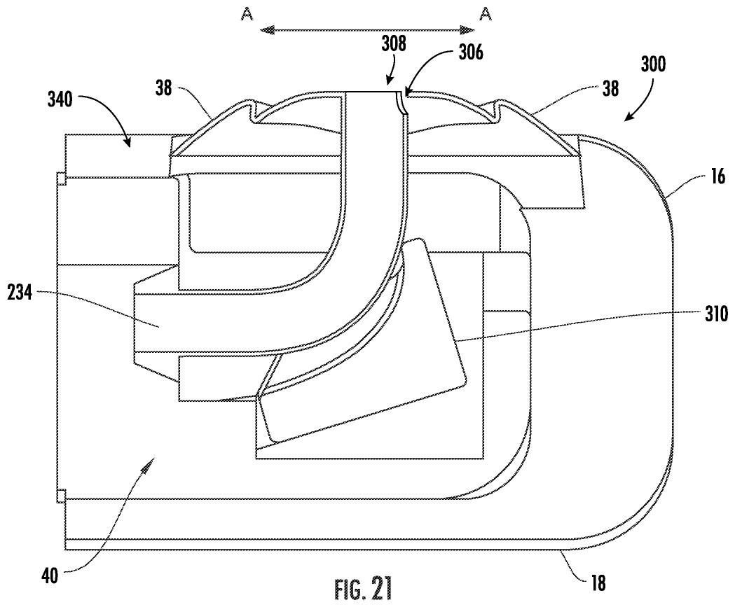

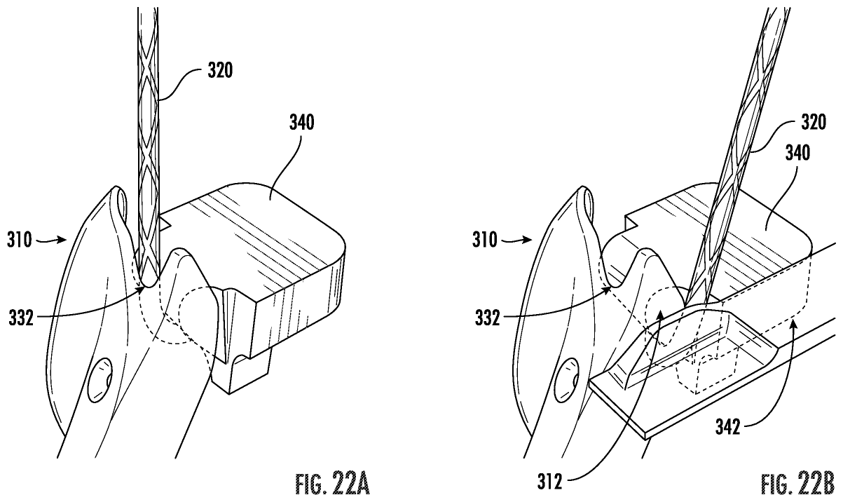

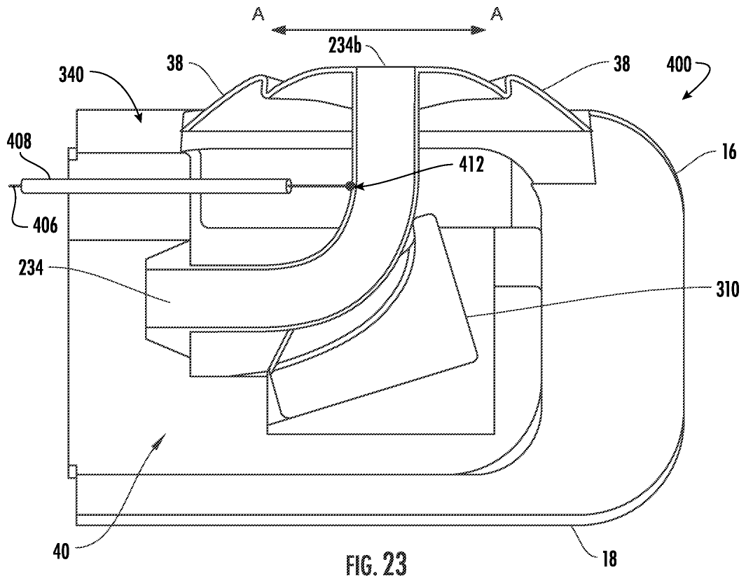

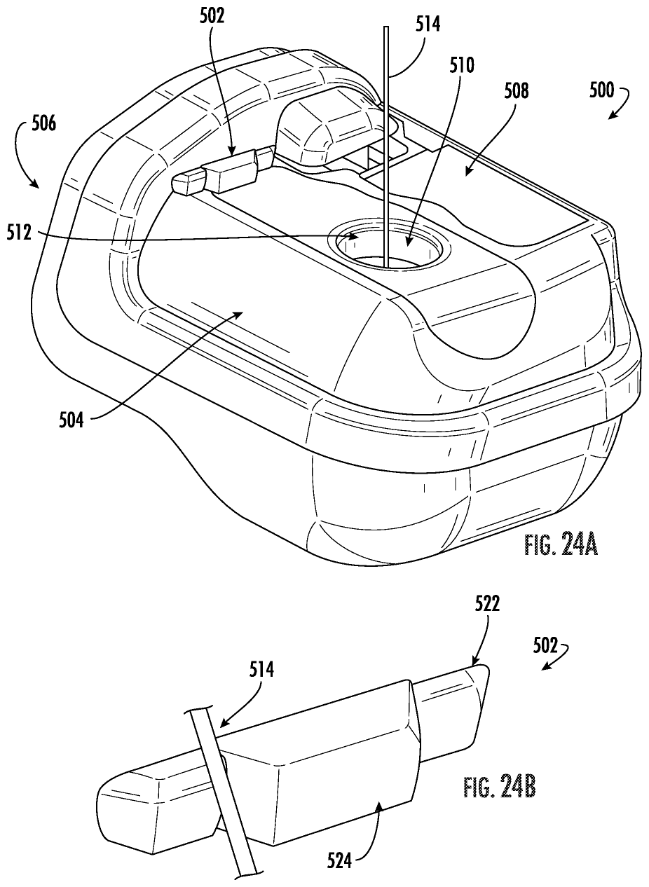

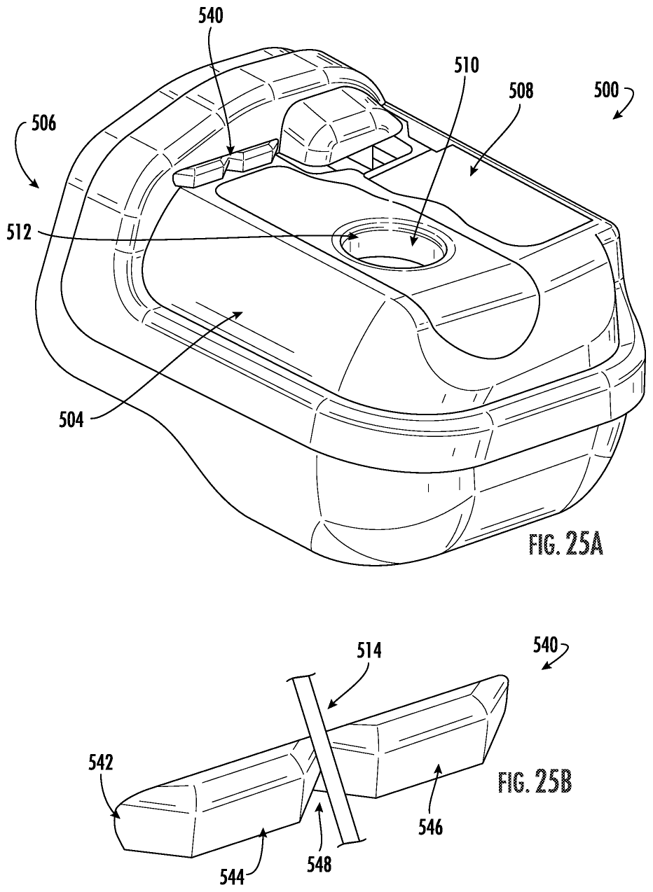

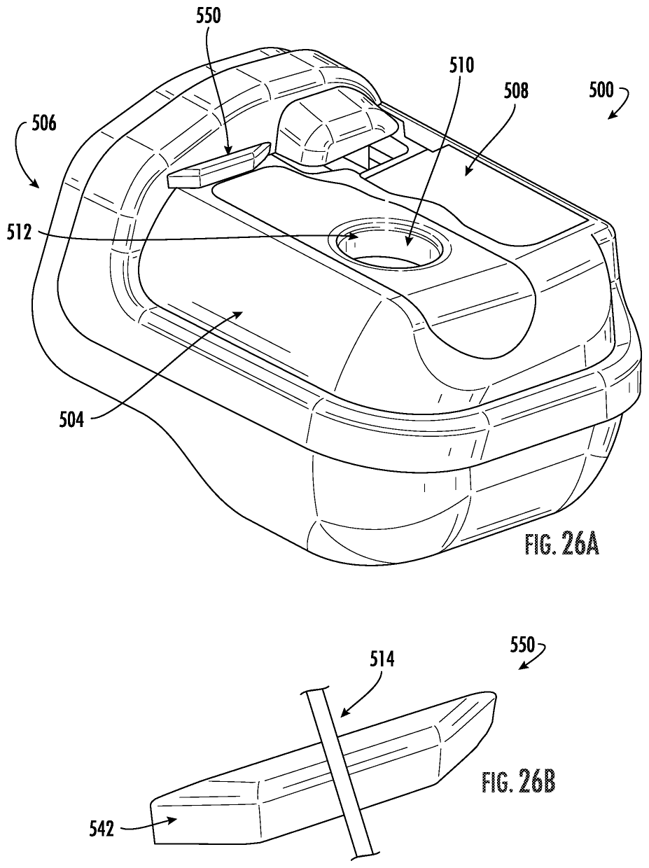

In FIG. 18, an alternative embodiment is shown for locking an instrument 200 in place. In this embodiment, the working channel extension 234 is raised to a point in which the instrument 200 in the working channel extension 234 is compressed against a lock 180 on the device 100, causing a change in the angle of exit of the working channel extension 234 and locking the instrument 200 in a fixated place in the working channel extension 234.

In FIG. 19, an alternative embodiment of the working channel extension 234 is shown with a flange 268 for attaching the working channel extension to the membrane material 38 that is part of the device 10.

In FIG. 20, an alternative embodiment of working channel extension 234 for a coupler device is shown that includes an indentation or groove 306 in a distal open end 308 for receiving an instrument (not shown) passing through an internal lumen 304 of working channel extension 234. Groove 306 is dimensioned to constrain and/or secure an instrument in place after it has passed through working channel extension 234 so that it does not move around at the target site in the patient. Groove 306 can, for example, be used by an operator to fixate an instrument and/or to fixate a guidewire while another instrument is exchanged over the guidewire.

The instrument may include a guidewire, an endoscopic mucosal resection instrument, needle injector, Foley catheter, bipolar or monopolar electrosurgical or ultrasonic devices, snares, endoscopic staplers and other clamping or sealing instruments, arterial lines, drainage catheters, peripherally inserted central catheters, and other devices that penetrate and/or navigate in the body. The instrument may be configured to advance through the working channel of the endoscope and the working channel extension or other passageway of the coupler device.

As shown, groove 306 preferably forms a substantially V shape, although it will be recognized by those skilled in the art that other shapes are envisioned. For example, the groove may be beveled or angled, or form a partial cylinder, cone, square, triangular, U-shape or other suitable configuration. Groove 306 provides a firmer grip on the instrument to lock the instrument in place within working channel extension 234.

Groove 306 may be formed directly into working channel extension 234. Alternatively, groove 306 may be formed from a separate component that is attached to the distal end of working channel extension 234. In this latter embodiment, groove 306 may, for example, be formed from a biocompatible component that can be removably attached to working channel extension 234 prior to, or during, a procedure on a patient.

In one embodiment, working channel extension 234 optionally includes one or more bevels, projections or protrusions 302 extending into internal lumen 304 to stricture or narrow the passage in the distal direction and to further define groove 306. Protrusions 302 serve to form a slope within the internal lumen 304 of working channel extension 234 to guide the instrument into groove 306. Protrusions 302 may extend around the entire circumference of internal lumen 304, or they may only be formed on one portion of internal lumen 304. For example, protrusions 302 may form a semi-hemisphere around a portion of lumen 304.

Protrusions 302 may be formed directly into working channel extension 234 during manufacturing through a variety of techniques well-known in the art. Alternatively, protrusions 302 may be a separate component that is attached to working channel extension 234. Protrusions 302 and groove 306 may be formed together or they may be separate components. The separate component(s) may be removably attached to working channel extension 234 so that it can be used when needed during a procedure, and then removed when no longer desired. Alternatively, protrusions 302 and/or groove 306 may be permanently affixed to working channel extension 234. In certain embodiments, protrusions 302 and groove 306 are formed from a metal, such as stainless steel or titanium or a plastic material, such as PMMA, polycarbonate, polyethylene or the like, or any other suitable biocompatible material.

The surface of protrusions 302 may be formed from a material that increases friction with the instrument to facilitate the grip therebetween.

Alternatively, the inner surfaces of protrusions 302 may include surface features, such as abrasions, a roughened surface or the like that take advantage of, for example, a guidewire' s reactive force to secure the guidewire to working channel extension 234.

Referring now to FIG. 21, coupler device 300 may include an elevator 310 for articulating working channel extension 234 (alternatively, the elevator may be part of scope 40). Working channel extension 234 may also include a groove 306 at the open distal end 308, as shown in FIG. 20. In this embodiment, the working channel extension 234 does not include any protrusions within the inner lumen of working channel extension 234, although such protrusions could be included as described in FIG. 20.

In use, when elevator 310 is actuated, the flexible working channel extension 234 of the coupler device moves or adjusts to this actuation, along the direction A—A, as discussed above. As elevator 310 moves the flexible working channel extension 234, it slightly compresses the internal diameter of working channel extension 234 to secure the instrument within groove 306 or to secure the instrument in working channel extension 234. Alternatively, coupler device 300 may further include a stop member (not shown) within main body 340 positioned such that working channel extension 234 engages the stop member as elevator 310 moves working channel extension 234. This engagement causes a slight compression of one of the walls of working channel extension 234 to reduce its inner diameter and secure an instrument therein. Alternatively, the stop member may be coupled to an actuator configured to translate or advance the stop member against working channel extension 234. In this latter embodiment, the instrument may be secured within working channel extension 234 at any location that it is movable along the direction A-A.

Alternatively, coupler device 300 may be constructed without protrusions 302 or groove 306. In this embodiment, elevator 310 articulates the flexible working channel extension against another fixed member of coupler device 300 in order to secure the instrument therein.

FIGS. 22A and 22B illustrate another embodiment wherein a coupler device according to the present disclosure includes an elevator 310 for articulating an instrument, such as a guidewire 320. The remainder of the coupler device 300 is not shown in these figures. In this embodiment, coupler device 300 may, or may not, include a working channel extension. For example, coupler device 300 may comprise an open area or passageway between the distal end of the working channel of scope 40 and elevator 310. This open area or passageway allows for advancement of an instrument from the scope working channel to the elevator 310. The elevator 310 can be rotated to articulate the instrument and change its exit angle from coupler device 300. The elevator 310 may be rotated by a variety of different actuators, as discussed above.

As shown in FIG. 22A, elevator 310 includes a groove 332 near its distal end for receiving the instrument (e.g., a guidewire 320). As in previous embodiments, groove 332 may have a substantially V-shape or any other suitable shape for securing guidewire 320 therein. Elevator 310 may be articulated such that it engages a stop member 340, which may be part of the main body of coupler device 300, or it may be a separate component of device 300. Alternatively, elevator 310 may be designed to engage the distal end portion of the scope in which case the coupler device will not include stop member 340. As elevator 310 engages stop member 340 or the distal end of the scope, the guidewire 320 is secured within groove 332.

FIG. 22B illustrates an alternative feature of the coupler device of the present disclosure. As shown, an instrument, such as guidewire 320, may be advanced along a side surface 312 of elevator 310. A stop member 342 is provided laterally of elevator 310 to secure guidewire 320 between side surface 312 and stop member 342. Lateral stop member 342 may be translated or articulated into position to engage guidewire 320 therebetween. Alternatively, elevator 310 may be designed to articulate into position such that guidewire 320 is secured between side surface and lateral stop member 342. In yet another embodiment, coupler device 300 does not include lateral stop member 342 and guidewire 320 is secured between elevator 310 and a side wall (not shown) of coupler device 300. In this embodiment, coupler device 300 may secure two separate instruments at the same time (i.e., one within groove 332 and one along the side surface 312).

FIG. 23 illustrates yet another embodiment of a coupler device 400 according to the present disclosure. As shown, coupler device includes a working channel extension 234 having a proximal end configured for coupling to a distal end of a working channel of an endoscope and an open distal end 234b. In this embodiment, coupler device 400 or the endoscope may, or may not, include a separate elevator 310 for articulating working channel extension 234. A cable 406 extends through an outer sheath 408 and has a distal end 412 coupled to one portion of working channel extension 234. Cable 406 is configured for advancement in the distal direction to compress the portion of working channel extension 234, thereby reducing its inner diameter and securing an instrument passing therethrough. Alternatively, cable 406 may remain stationary as elevator 310 moves working channel extension 234 against cable 406 to thereby compress a portion of working channel extension 234. Working channel extension 234 may, or may not, include one or more protrusions or grooves in its internal lumen to facilitate the securement of the instrument.

FIGS. 24A and 24B illustrate another embodiment of a coupler device 500 having a locking element 502 according to the present disclosure. As shown, coupler device 500 includes a main body 504 with a proximal end 506 configured for attaching onto a working end of an endoscope (not shown), thereby extending the working end portion of the scope. Similar to previous embodiments, main body 504 has an upper surface 508 that may include a lens and light guide and a scope washer opening. Upper surface 508 may further include an open area over the lens and light guide and the scope washer opening to facilitate viewing the surgical site and to allow egress of fluid from the scope washer opening into the surgical site (and/or egress of air that may be passed over the light guide to dry the camera or that may be passed into the surgical site to insufflate a portion of the site).

In addition, the upper surface 508 includes a flexible working channel region that includes a flexible working channel extension 510 that has a proximal end (not shown) configured for attachment to the working channel of the endoscope and a distal opening 512 for passing instruments 514, such as a guidewire or the like, through working channel extension 510 into the patient. Similar to previous embodiments, coupler device 500 may further include an elevator or other mechanism for articulating working channel extension 510 so as to change the angle that the instrument 514 exits distal opening 512. Alternatively, coupler device 500 may be configured to work with an elevator on the endoscope, as discussed above.

As shown, coupler device 500 further includes a locking element 502 positioned on upper surface 508 for constraining and/or locking instrument 514 in place. Locking element 502 comprises an elongate body 522 extending transverse to, or substantially perpendicular to, the longitudinal axis of main body 504. Locking element 502 further includes a central protrusion 524 extending outward from elongate body 522 and positioned to lock instrument 514 between elongate body 522 and working channel extension 510 or to deflect instrument 514 to the side of central protrusion 524 (as shown in FIG. 24B). Locking element 502 may comprise any suitable biocompatible material that is rigid or at least semi-rigid, such as an exposed plastic, polycarbonate, acrylic and the like. Alternatively, locking element 502 may comprise a more elastic material, such as a thermoplastic elastomer (TPE) and the like.

In use, working channel extension 510 is articulated in a direction generally parallel to the longitudinal axis of main body 504 such that the instrument 514 is compressed against elongate body 522 on one side of central protrusion 524 of locking element 502. In one embodiment, raising the elevator causes working channel extension 510 to temporarily compress and lock the instrument 514. Alternatively, as the elevator is raised, the TPE over the elevator may directly contact instrument 514 and compress instrument 520 against locking element 502. In certain embodiments, multiple instruments may be locked on either side of central protrusion 524.

FIGS. 25A and 25B illustrate another embodiment of a locking element 540 for a coupler device 500. As shown, locking element 540 comprises an elongate body 542 with two protrusions 544, 546 that define a central groove 548 therebetween. In this embodiment, instrument 514 is locked into central groove 548 when working channel extension 510 is compressed against locking element 540. Similar to previous embodiments, groove 548 may be substantially V-shaped, or any other suitable shape that facilitates securing instrument 514 therein.

FIGS. 26A and 26B illustrate yet another embodiment of a locking element 550 for a coupler device 500. As shown, locking element 550 comprises an elongate body 542 extending transverse to the longitudinal axis of endoscope and coupler device 500. In this embodiment, locking element 550 does not contain any protrusions extending from elongate body 542. In use, the elevator is raised to articulate working channel extension 510 such that instrument 514 is locked against elongate body 542.

The coupler devices and endoscopes of the present disclosure may be used in a variety of different kits that include other medical devices, instruments, accessories, balloons, endoscopic positioning systems, guidewires, dilation catheters, cannulas, cutting devices (e.g., sphincterotomes or papillotomes), choledochoscopes, stone entrapment and extraction devices, polyp or tissue removal devices, snares, stents (e.g., biliary stents), disposable valves, bit blocks, anatomic support bands or other devices. A more complete description of kits for use with the present disclosure can be found in commonly-assigned, co-pending U.S. Patent

Applications Nos. 16/717,202 and 16,717, 804, both filed on December 17, 2019, the completed disclosures of which are hereby incorporated by reference for all purposes.

Hereby, all issued patents, published patent applications, and non-patent publications that are mentioned in this specification are herein incorporated by reference in their entirety for all purposes, to the same extent as if each individual issued patent, published patent application, or non-patent publication were specifically and individually indicated to be incorporated by reference.

Other embodiments will be apparent to those skilled in the art from consideration of the specification and practice of the embodiment disclosed herein. It is intended that the specification and examples be considered as exemplary only, with a true scope and spirit of the embodiment being indicated by the following claims. Amendments to the Claims:
This listing of claims will replace all prior versions, and listings of claims in the application:
Please amend claims 7, 13, 15 and 18 and cancel claims 8, 9, 14, 19, 21, 22, 23 and 30-44. Claims 1-7, 10-13, 15-18, 20 and 14-29 remain pending. 1 (Original) A device for use with an endoscope, the device comprising:
a main body comprising a visualization section for allowing viewing of tissue by the endoscope and a proximal end configured for attachment to a distal end portion of the endoscope;
an instrument channel within the main body and configured to be in operational proximity to a working channel in the endoscope when the proximal end of the main body is attached to the distal end portion of the endoscope; and
wherein the device is configured to constrain an instrument passing through the instrument channel.

2. The device of claim 1, wherein the instrument channel is a working channel extension within the main body, the working channel extension having an open distal end and a proximal end that is configured for attachment to the working channel of the endoscope.

3. The device of claim 2, wherein the device is configured to compress a portion of the working channel extension against the instrument to secure the instrument in place within the working channel extension.

4. The device of claim 2, wherein the open distal end is oriented at an angle relative to a longitudinal axis of the endoscope working channel, the device being configured to secure the instrument in position at said angle.

5. The device of claim 4, wherein the device is configured to allow movement of the instrument while the instrument is secured at said angle.

6. The device of claim 1 further comprising an elevator within the main body for angular adjustment of the instrument channel.

7. The device of claim 6 further comprising a locking element on an outer surface of the main body, wherein the elevator is configured to secure an instrument against the locking element, wherein the locking element comprises a main body extending transverse to the longitudinal axis of the working channel and further comprises one or more protrusions extending from the main body to define a groove for securing the instrument.

8. (canceled)

9. (canceled)

10. The device of claim 6, wherein the elevator is configured to compress a portion of the instrument channel upon said angular adjustment to secure the instrument in position.

11. The device of claim 1, wherein the instrument channel defines a groove at the distal end for receiving the instrument.

12. The device of claim 11, wherein the groove has a substantially V-shape.

13. The device of claim 11, further comprising one or more protrusions extending into the instrument channel and an actuator for compressing the instrument channel to secure the instrument within the groove.

14. (canceled)

15. The device of claim 13, wherein the protrusions are removably coupled to the instrument channel.

16. The device of claim 1 further comprising a mechanism for articulating an instrument passing through the endoscope.

17. The device of claim 2, wherein the working channel extension is flexible and capable of angular adjustment by actuation of the endoscope.

18. The device of claim 17 further comprising a stop member positioned to engage a portion of the working channel extension, wherein the working channel extension is configured to partially compress upon engagement with the stop member, wherein the working channel extension is configured to engage the stop member upon angular adjustment of the working channel extension.

19. (canceled)

20. The device of claim 2 further comprising an actuator coupled to a portion of the working channel extension and configured to compress said portion to reduce a diameter of the working channel extension.

21. (canceled)

22. (canceled)

23. (canceled)

24. A kit for use in a procedure on a patient, the kit comprising:
an endoscope having an optical element and a working channel for receiving an instrument; and
a coupler device comprising:
a main body comprising a visualization section for allowing viewing of tissue by the endoscope and a proximal end configured for attachment to a distal end portion of the endoscope;
a passageway within the main body having an open distal end and a proximal end in operational proximity to the working channel of the endoscope when the proximal end of the coupler device is attached to the distal end portion of the endoscope;
and wherein the coupler device is configured to constrain an instrument passing through the passageway.

25. The kit of claim 24 further comprising the instrument.

26. The kit of claim 25, wherein the instrument comprises a guidewire.

27. The kit of claim 24, wherein the coupler device is configured to secure the instrument in place.

28. The kit of claim 24, wherein the open distal end is oriented at an angle relative to the endoscope working channel, the coupler device being configured to secure the instrument into position at said angle.

29. The kit of claim 24, wherein the coupler device is configured to allow translational movement of the instrument while the instrument is secured at said angle.

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)
